(19) United States
(12) Patent Application Publication
(10) Pub. No.: US 2026/0273248 A1
(43) Pub. Date: Sep. 17, 2026

(54) FLUSH THROUGH DILATOR AND RELATED METHODS

(71) Applicant: NeuroFine Corp., Miramar, FL (US)

(72) Inventor: Brad D. Aurilia, Coral Springs, FL (US)

(21) Appl. No.: 19/468,208

(22) Filed: Feb. 3, 2026

Related U.S. Application Data
(63) Continuation of application No. PCT/US2024/040149, filed on Jul. 30, 2024.
(60) Provisional application No. 63/531,893, filed on Aug. 10, 2023.

(51) Int. Cl. A61M 29/00; A61M 25/09

(52) U.S. Cl. CPC A61M 29/00; A61M 25/09041; A61M 2210/12

(57) ABSTRACT

A dilator includes a shaft, a flushing lumen, and an entrance port. The shaft includes a proximal end portion and a distal end portion. The flushing lumen extends longitudinally through the shaft from the proximal end portion to the distal end portion and is configured for injecting a liquid through the dilator. The entrance port is in the proximal end portion of the shaft and is in fluid communication with the flushing lumen.

### CROSS REFERENCE TO RELATED APPLICATIONS

This application is a continuation of PCT Patent Application No. PCT/US2024/040149 filed on Jul. 30, 2024, which claims the priority to U.S. Provisional Patent Application Serial No. 63/531,893, filed Aug. 10, 2023, the disclosures of which are incorporated herein by reference in their entirety.

### TECHNICAL FIELD

The present application generally relates to closure devices and methods for sealing punctures or other openings in blood vessels, such as the common carotid artery made during a direct access approach for neurological therapy. The devices and methods may be used in the course of other therapies or surgical procedures as well.

### BACKGROUND

Traditionally, dilators are used in conjunction with guidewires and introducer sheaths to access blood vessels. A guidewire is inserted through a central lumen of the dilator and the dilator is inserted through the introducer sheath. Generally, a blood vessel is pierced with a needle creating an opening in the blood vessel. The guidewire is inserted through the opening and into the blood vessel. The dilator is directed along the guidewire to dilate the opening in the blood vessel so that a portion of an introducer sheath can pass through the opening and access the inside of the blood vessel. The distal tip of the dilator is tapered to expand the opening in the blood vessel as it is advanced through it. Once the introducer sheath has accessed the inside of the blood vessel, the surgeon may want to inject a contrast media or other liquid into the blood vessel in order to better observe the procedure, for example. Traditionally, in order to inject the contrast media through the injection port of the introducer sheath the guidewire and/or the dilator must be removed from the introducer sheath. Neither of these options are ideal if the guidewire and/or the dilator need to be re-inserted to continue the procedure. Removing the guidewire and/or the dilator from the introducer sheath may prolong the procedure and/or disrupt the introducer sheath and /or the patient.

To overcome these limitations, some dilators include a second lumen that c an be used for flushing or injecting a contrast media. These dilators include a separate flushing port since the second lumen is not configured to be in fluid communication with the flushing port of the introducer sheath.

Accordingly, and despite the various advances already made in this field, there is a need for further improvements to devices, systems and methods related to flushable dilators .

### SUMMARY

Generally, a dilator is provided. The dilator includes a shaft, a flushing lumen, and an entrance port. The shaft has a proximal end portion and a distal end portion. The flushing lumen extends longitudinally through the shaft from the proximal end portion to the distal end portion. The flushing lumen is configured for injecting a liquid through the dilator. The entrance port is located in the proximal end portion of the shaft and is in fluid communication with the flushing lumen.

In some embodiments, the dilator may be configured for use with an introducer sheath. The entrance port may be configured to be in fluid communication with the introducer sheath. The entrance port may be configured to be in fluid communication with a flushing port of the introducer sheath. The dilator may include a lock located on the proximal end portion of the shaft and configured to engage the introducer sheath and couple the dilator to the introducer sheath. The dilator may have a hub located on the proximal end portion of the shaft and including a lock configured to engage the introducer sheath and couple the dilator to the introducer sheath. The dilator may include an exit port located in the distal end portion of the shaft and in fluid communication with the flushing lumen. The dilator may have a first exit port and a second exit port located in the distal end portion of the shaft and in fluid communication with the flushing lumen. The dilator may have a tip portion located on the distal end portion of the shaft. The tip portion may include an exit port in fluid communication with the flushing lumen. The tip portion may include a first exit port and a second exit port in fluid communication with the flushing lumen. The dilator may have a central lumen extending longitudinally through the shaft from the proximal end portion to the distal end portion. The central lumen may be configured to receive a guidewire therethrough.

In alternate or additional aspects, the dilator may have a first flushing lumen and a second flushing lumen. The dilator may have a first entrance port and a second entrance port in the proximal end portion of the shaft. The first entrance port may be in fluid communication with the first flushing lumen. The second entrance port may be in fluid communication with the second flushing lumen. The dilator may have a first exit port and a second exit port located in the distal end portion of the shaft. The first exit port may be in fluid communication with the first flushing lumen. The second exit port may be in fluid communication with the second flushing lumen. The dilator may have a tip portion located on the distal end portion of the shaft. The tip portion may include a first exit port in fluid communication with the first flushing lumen and a second exit port in fluid communication with the second flushing lumen. The dilator may have a central axis, and the first flushing lumen may be located opposite the second flushing lumen along the central axis.

An alternative dilator is provided. The alternative dilator includes a shaft, a flushing lumen, an entrance port, and a central lumen. The shaft includes a proximal end portion and a distal end portion. The flushing lumen extends longitudinally through the shaft from the proximal end portion to the distal end portion and is configured for injecting a liquid through the dilator. The entrance port is located in the proximal end portion of the shaft and is in fluid communication with the flushing lumen. The central lumen extends longitudinally through the shaft from the proximal end portion to the distal end portion.

In some embodiments, the central lumen is configured to receive a guidewire therethrough. The dilator may be configured for use with an introducer sheath. The entrance port may be configured to be in fluid communication with the introducer sheath. The entrance port may be configured to be in fluid communication with a flushing port of the introducer sheath. The dilator may include a lock located on the proximal end portion of the shaft and configured to engage the introducer sheath and couple the dilator to the introducer sheath. The dilator may have a hub located on the proximal end portion of the shaft and including a lock configured to engage the introducer sheath and couple the dilator to the introducer sheath. The dilator may have an exit port located in the distal end portion of the shaft and in fluid communication with the flushing lumen. The dilator may have a first exit port and a second exit port located in the distal end portion of the shaft and in fluid communication with the flushing lumen. The dilator may include a tip portion located on the distal end portion of the shaft. The tip portion may include an exit port in fluid communication with the flushing lumen. The tip portion may include first and second exit ports in fluid communication with the flushing lumen.

In alternate or additional aspects, the dilator may have a first flushing lumen and a second flushing lumen. The dilator may have a first entrance port in the proximal end portion of the shaft and in fluid communication with the first flushing lumen. The dilator may have a second entrance port in the proximal end portion of the shaft and in fluid communication with the second flushing lumen. The dilator may have a first exit port located in the distal end portion of the shaft and in fluid communication with the first flushing lumen. The dilator may have a second exit port located in the distal end portion of the shaft and in fluid communication with the second flushing lumen. The dilator may include a tip portion located on the distal end portion of the shaft. The tip portion may include a first exit port in fluid communication with the first flushing lumen. The tip portion may include a second exit port in fluid communication with the second flushing lumen. The dilator may have a central axis and the first flushing lumen may be located opposite the second flushing lumen along the central axis.

An alternative dilator is provided. The alternative dilator includes a shaft, a first flushing lumen, a second flushing lumen, a first entrance port, a second entrance port, and a central lumen. The shaft includes a proximal end portion and a distal end portion. The first and second flushing lumens extend longitudinally through the shaft from the proximal end portion to the distal end portion and are configured for injecting a liquid through the dilator. The first and second entrance ports are in the proximal end portion of the shaft and are in fluid communication with the first and second flushing lumens respectively. The central lumen extends longitudinally through the shaft from the proximal end portion to the distal end portion.

In some embodiments, the central lumen is configured to receive a guidewire therethrough. The dilator may be configured for use with an introducer sheath. The first and second entrance ports may be configured to be in fluid communication with the introducer sheath. The first and second entrance ports may be configured to be in fluid communication with a flushing port of the introducer sheath. The dilator may include a lock located on the proximal end portion of the shaft and configured to engage the introducer sheath and couple the dilator to the introducer sheath. The dilator may include a hub located on the proximal end portion of the shaft and include a lock configured to engage the introducer sheath and couple the dilator to the introducer sheath. The dilator may include first and second exit ports located in the distal end portion of the shaft and in fluid communication with the first and second flushing lumens respectively. The dilator may have a tip portion located on the distal end portion of the shaft. The tip portion may include first and second exit ports in fluid communication with the first and second flushing lumens respectively. The dilator may have a central axis. The first flushing lumen may be located opposite the second flushing lumen along the central axis.

A method of using a dilator is provided and includes introducing a dilator into an introducer sheath, and injecting a liquid into the introducer sheath and through the dilator. The method may include coupling the dilator to the introducer sheath. The method may include injecting the liquid into an injection port of the introducer sheath. The liquid may be a contrast media. The method may include introducing a guidewire into a lumen of the dilator.

A method of manufacturing a dilator is provided and includes forming a shaft including a proximal end portion and a distal end portion, forming a flushing lumen into the shaft extending longitudinally through the shaft from the proximal end portion of the shaft to the distal end portion of the shaft, and forming an entrance port in the proximal end portion of the shaft and in fluid communication with the flushing lumen.

In some embodiments, the method may include sealing a proximal end of the flushing lumen. Sealing the proximal end of the flushing lumen may include sealing the proximal end of the flushing lumen with epoxy. The method may include forming an exit port located in the distal end portion of the shaft and in fluid communication with the flushing lumen. The method may include forming a tip portion on the distal end portion of the shaft. The method may include forming an exit port in the tip portion of the shaft and in fluid communication with the flushing lumen. Forming a flushing lumen into the shaft may include forming first and second flushing lumens. The method may include sealing a proximal end of the first and second flushing lumens. Sealing the proximal ends of the first and second flushing lumens may include sealing the proximal ends of the flushing lumens with epoxy. Forming an entrance port may include forming first and second entrance ports in fluid communication with the first and second flushing lumens respectively. The method may include forming first and second exit ports located in the distal end portion of the shaft and in fluid communication with the first and second flushing lumens respectively. The method may include forming a tip portion on the distal end portion of the shaft. The method may include forming first and second exit ports in the tip portion of the shaft and in fluid communication with the first and second flushing lumens respectively. The method may include forming a central lumen into the shaft. The central lumen may be configured to receive a guidewire therethrough. The method may include coupling a hub to the proximal end portion of the shaft. The method may include coupling a lock to the proximal end portion of the shaft configured to engage an introducer sheath and couple the dilator to the introducer sheath.

Any of the features and functions described herein may be applied to any of the disclosed embodiments or methods. Additional features and advantages of the inventive aspects disclosed herein will become more apparent upon review of the following detailed description taken together with accompanying drawings of the illustrative and exemplary embodiments.

### BRIEF DESCRIPTION OF THE DRAWINGS

FIG. 1 is a perspective view of an illustrative flush through dilator configured for use with an introducer sheath.

FIG. 2 is a perspective view of a shaft of the flush through dilator of FIG. 1.

FIG. 3 is a section view of the shaft of the flush through dilator of FIGS. 1 and 2.

FIG. 4 is a perspective view of the flush through dilator of FIG. 1 inserted into an introducer sheath.

FIG. 5 is a sectional view of the flush through dilator and introducer sheath of FIG. 4.

### DETAILED DESCRIPTION

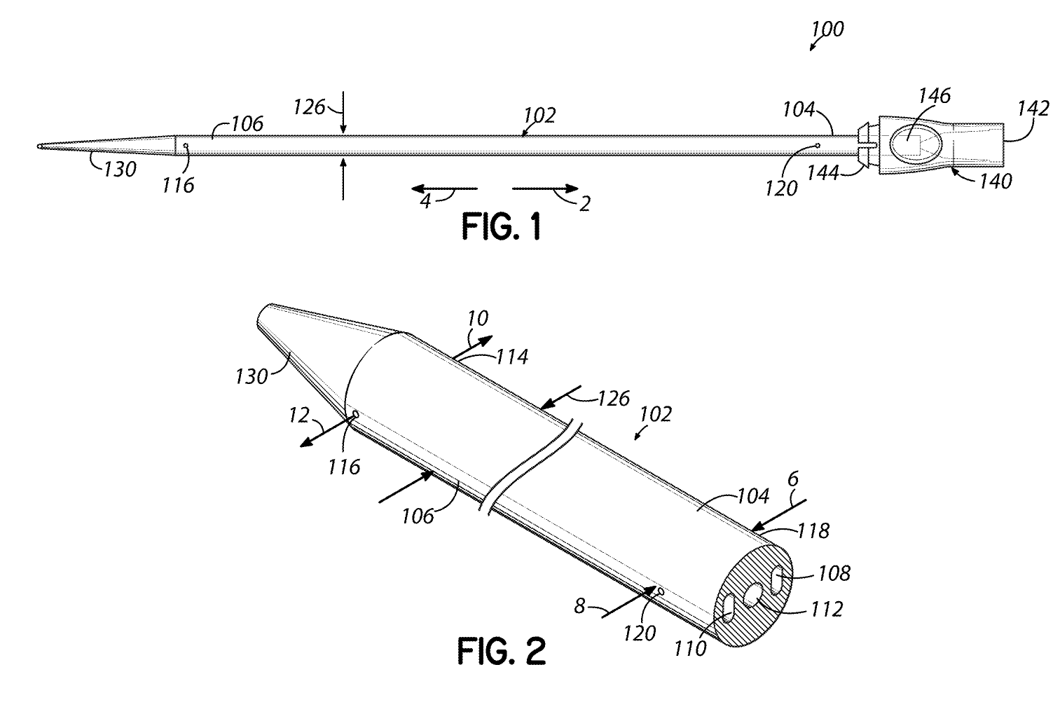

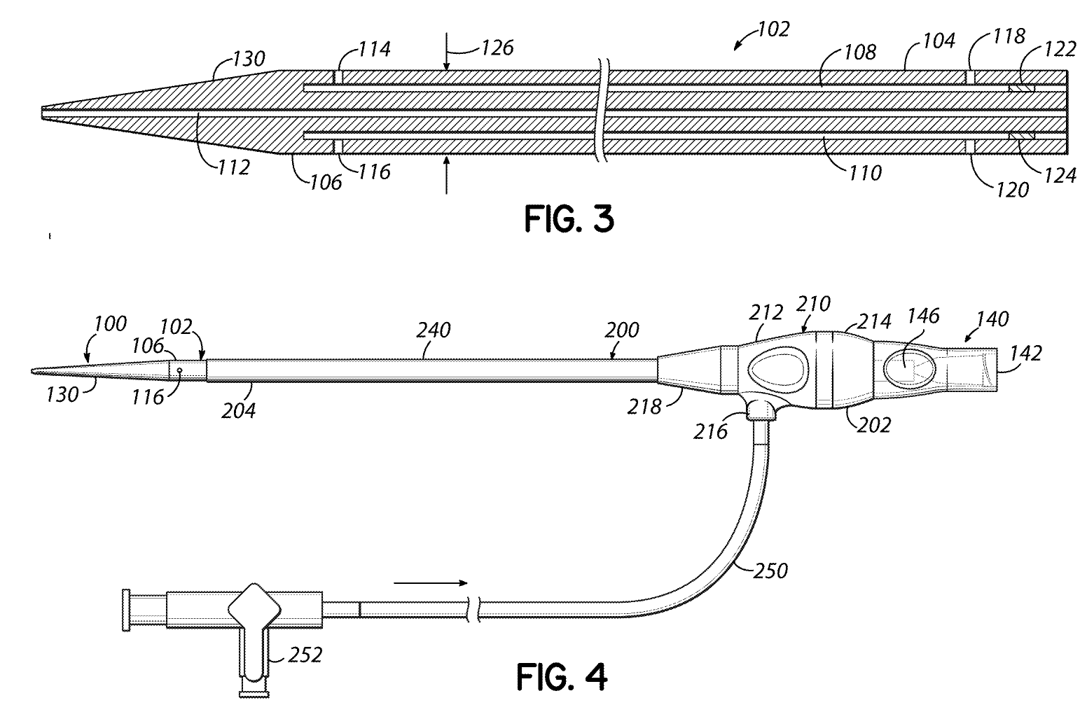

FIG. 1 is a perspective view of an illustrative flush through dilator 100 configured for use with an introducer sheath, according to at least some aspects of the present disclosure. In this illustrative embodiment, the dilator 100 includes a n elongated shaft 102, a tip portion 130, and a dilator hub 140. FIG. 2 is a perspective view of the shaft 102 and FIG. 3 is a section view of the shaft 102, all according to at least some aspects of the present disclosure.

The elongated shaft 102 has a proximal end portion 104 and a distal end portion 106. The tip portion 130 is located on the distal end portion 106 of the shaft 102, and the hub 140 is coupled to the proximal end portion 104 of the shaft 102. As used herein to describe various embodiments from the perspective of a user of a surgical device, “proximal” may refer to a direction generally towards the user of the device, while “distal” may refer to a direction generally away from the user of the device. Similarly, in the context of a surgical device inserted into a patient’s body and from the perspective of a user of the device, “proximal” may refer to a direction generally away from the patient’s body, and “distal” may refer to a direction generally towards the patient’s body. For reference, arrow 2 points generally proximally and arrow 4 points generally distally.

Referring to FIGS. 2 and 3, in this illustrative embodiment, the shaft 102 has first and second flushing lumens 108, 110, and a central lumen 112 extending longitudinally through the shaft 102 from the proximal end portion 104 to the distal end portion 106. The central lumen 112 defines a central axis of the shaft 102. The first flushing lumen 108 is located opposite the second flushing lumen 110 along the central axis of the shaft 102 . Some embodiments may have one flushing lumen while other embodiments may have more than two flushing lumens. The dilator 100 includes exit ports 114, 116 located in the distal end portion 106 of the shaft 102. The exit ports 114, 116 are connected to and in fluid communication with the flushing lumens 108, 110 respectively. In some embodiments, the exit ports 114, 116 may be in the tip portion 130 of the dilator 100. Some embodiments may have one exit port while other embodiments may have more than two exit ports. The dilator 100 includes entrance ports 118, 120 in the outer diameter 126 of the proximal end portion 104 of the shaft 102. The entrance ports 118, 120 are connected to and in fluid communication with the flushing lumens 108, 110 respectively. Some embodiments may have one entrance port while other embodiments may have more than two entrance ports. As shown in FIG. 3, the flushing lumens 108, 110 are sealed with plugs 122, 124 in the proximal end portion 104 of the shaft 102. The proximal ends of the flushing lumens 108, 110 are sealed to prevent liquid from flowing in the proximal direction and exiting from the proximal ends of the flushing lumens 108, 110. The plugs 122, 124 sealing the proximal ends of the flushing lumens 108, 110 may be epoxy, for example. The plugs 122, 124 may be any material that effectively seals the proximal ends of the flushing lumens 108, 110. In some embodiments, the shaft 102 may have one flushing lumen in fluid communication with one or more exit ports and/or entrance ports.

Referring to FIG. 1, in this illustrative embodiment, the tip portion 130 is located on the distal end portion 106 of the shaft 102. The tip portion 130 is tapered enabling the dilator to dilate tissue, such as the wall of a blood vessel for example, as the tip portion 130 is advanced through the tissue.

Referring again to FIG. 1, the dilator hub 140 is coupled to the proximal end portion 104 of the shaft 102. The dilator hub 140 includes a dilator hub lumen 142 that is aligned with the shaft central lumen 112, see FIG. 2. The shaft central lumen 112 is configured for use with a guidewire and the hub lumen 142 is configured to facilitate the introduction of a guidewire into the shaft central lumen 112. The hub 140 has a dilator lock 144 configured to couple the dilator 100 to an introducer sheath 200, see FIG. 4. In this illustrative embodiment, the dilator lock 144 is one or more locking tabs. In alternative embodiments, the dilator lock 144 may be any device configured to couple the dilator 100 to an introducer sheath 200. In some embodiments, the outer surface of the hub 210 may include indentions 146, or other features, on the sides of the hub 140 to improve the ergonomics of the hub 140 and/or aid in coupling the dilator 100 with an introducer sheath 200, for example .

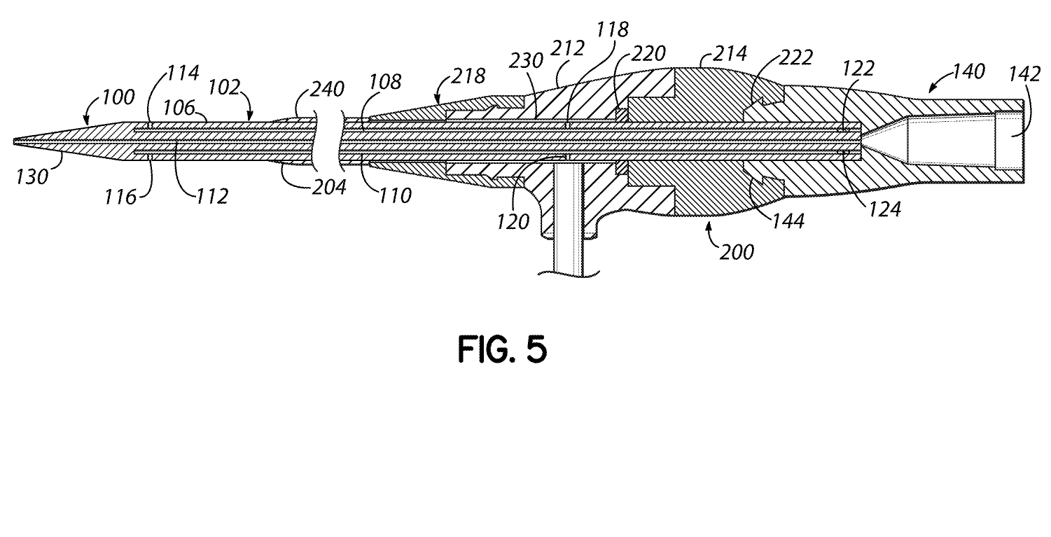

FIG. 4 is a perspective view of the illustrative dilator 100 inserted into an introducer sheath 200 and FIG. 5 is a sectional view of the illustrative dilator 100 inserted into the introducer sheath 200, all according to at least some aspects of the present disclosure.

In this illustrative embodiment, the introducer sheath 200 is configured to access a blood vessel. The introducer sheath 200 has a proximal end 202, a distal end 204, a hub 210 , a central lumen 230 , and a sheath 240. The hub 210 includes a front hub body 212, a rear hub body 214, an injection port 216, a strain relief 218, a seal 220, and a introducer sheath lock 222 .

A user can access the introducer sheath central lumen 230 at the proximal end 202 of the hub 210 with guidewires, dilators, and interventional devices, for example without concern for bleed-back. The seal 220 is designed to seal against an outer surface of a device, such as the dilator 100, that is installed in the introducer sheath 200. The seal 220 prevents blood or other bodily fluids from flowing proximally through the central lumen 230 of the introducer sheath 200 and around the installed device. The seal 220 also prevents blood or other bodily fluids from flowing proximally through the central lumen 230 of the introducer sheath 200 when a device, such as a dilator 100, is not installed in the introducer sheath 200. The seal 220 remains in a closed position when a device, such as a dilator 100 for example, is not passing through the central lumen 230. The seal 220 eliminates the need for an external valve such as a Tuohy Borst valve, for example. The introducer sheath lock 222 located in the rear hub body 214 allows a device, such as a dilator 100 for example, to be coupled to the introducer sheath 200 prior to a procedure, such as a blood vessel dilation , for example . The introducer sheath lock 222 also allow s for the device, such as a dilator 100 , to be easily removed from the introducer sheath 200 when the procedure is complete.

Referring to FIGS. 4 and 5, the injection port 216 may be used to remove air from the introducer sheath 200 at the start of a procedure and before a device, such as the dilator 100, is installed in the introducer sheath 200 . The injection port 216 may be also be used to inject a contrast media or other liquids through the central lumen 230 of the introducer sheath 200 . In this illustrative embodiment, the introducer sheath 200 is shown with tubing 250 and a 3-way valve 252 connected to the injection port 216. The tubing 250 and 3-way valve 252 provide additional functionality to the injection port 216. When the dilator 100 is installed in the introducer sheath 200, as shown in FIGS. 4 and 5, the flushing lumens 108, 110 are in fluid communication with the injection port 216 of the introducer sheath 200. When traditional dilators are installed in the introducer sheath 200, a user cannot inject liquids into the injection port 216 due to the minimal clearance between the outer diameter of the dilator and the inner diameter of the introducer sheath central lumen 230. However, when the dilator 100 is installed in the introducer sheath 200, a user may inject a contrast media or other liquids into the injection port 216 due to the flushing lumens 108, 110. Liquid injected into the injection port 216 flows into the introducer sheath hub 210 and enters the entrance ports 118, 120 of the dilator 100. Arrows 6, 8 show a direction a flushing liquid would enter the entrance ports 118, 120, see FIG. 2 . The injected liquid flows through the entrance ports 118, 120, into the flushing lumens 108, 110, and out of the exit ports 114, 116 at the distal end of the dilator 100. Arrows 10, 12 show a direction a liquid would leave the exit ports 114, 116, see FIG. 2 . The introducer sheath seal 220 prevents the liquid from flowing proximally through the central lumen 230 of the introducer sheath 200.

An illustrative method of using the dilator 100 is described herein. The dilator 100 is inserted into the central lumen 230 of introducer sheath 200 (see FIG. 5) and the dilator lock 144 engages the introducer sheath lock 222 thereby coupling the dilator 100 to the introducer sheath 200. A guidewire is inserted into the dilator hub lumen 142 and through the central lumen 112 of the dilator 100. In this example, a blood vessel, and a suitable location for accessing the blood vessel, are identified. A needle and the guidewire are directed to the identified access location on the blood vessel. The blood vessel is pierced with the needle creating an opening in the blood vessel. The guidewire is inserted through the opening and into the blood vessel. The dilator 100 is directed along the guidewire to dilate the opening in the blood vessel so the distal end 204 of the introducer sheath 200 can access the inside of the blood vessel. At this point in the procedure the surgeon may want to inject a contrast media into the blood vessel in order to better observe the procedure , for example . With the guidewire in the central lumen 112 and the dilator 100 in the introducer sheath 200 the surgeon may inject a contrast media into the injection port 216. The injected liquid flows into the hub 210 and enters the entrance ports 118, 120 of the dilator 100. The injected liquid flows through the entrance ports 118, 120, into the flushing lumens 108, 110, and out of the exit ports 114, 116 at the distal end of the dilator 100. Injecting a contrast media at this point in the procedure may allow the surgeon to continue the procedure without first removing the guidewire and /or the dilator 100 from the introducer sheath 200. By not removing the guidewire and /or the dilator 100 from the introducer sheath 200 the surgeon may continue the procedure with minimal disruption to the introducer sheath 200 and the patient.

An illustrative method of manufacturing the dilator 100 is described herein. The shaft 102 of the dilator 100 may be extrusion formed and the first and second flushing lumens 108, 110 and the central lumen 112 may be formed as part of the extrusion process, for example. A tip portion 130 may be formed on the distal end portion 106 of the shaft 102 after the shaft 102 is extruded, for example. The proximal ends of the first and second flushing lumens 108, 110 may be sealed with epoxy, for example. Alternatively, the proximal ends of the first and second flushing lumens 108, 110 may be sealed with plugs 122, 124 made from any material that effectively seals the proximal ends of the flushing lumens 108, 110. The exit ports 114, 116 are formed in the distal end portion 106 of the shaft 102 and are connected to and in fluid communication with the flushing lumens 108, 110 respectively. In some embodiments, the exit ports 114, 116 may be formed in the tip portion 130. The entrance ports 118, 120 are form ed in the proximal end portion 104 of the shaft 102 and are connected to and in fluid communication with the flushing lumens 108, 110 respectively . The hub 140 is formed with a hub lumen 142 and dilator lock 144 and is coupled to the proximal end portion of the shaft 102 .

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A dilator configured for use with an introducer sheath, the dilator comprising:
a shaft including a proximal end portion and a distal end portion;
a flushing lumen extending longitudinally through the shaft from the proximal end portion to the distal end portion, and configured for injecting a liquid through the dilator; and
an entrance port in the proximal end portion of the shaft, the entrance port configured to be in fluid communication with the introducer sheath and in fluid communication with the flushing lumen.

2. The dilator of claim 1, wherein the entrance port is configured to be in fluid communication with a flushing port of the introducer sheath.

3. The dilator of claim 1, further comprising a lock located on the proximal end portion of the shaft and configured to engage the introducer sheath and couple the dilator to the introducer sheath.

4. The dilator of claim 1, further comprising a hub located on the proximal end portion of the shaft and including a lock configured to engage the introducer sheath and couple the dilator to the introducer sheath.

5. The dilator of claim 1, further comprising an exit port located in the distal end portion of the shaft and in fluid communication with the flushing lumen.

6. The dilator of claim 1, further comprising a first exit port and a second exit port located in the distal end portion of the shaft and in fluid communication with the flushing lumen.

7. The dilator of claim 1, wherein the dilator further comprises a tip portion located on the distal end portion of the shaft, and the tip portion includes an exit port in fluid communication with the flushing lumen.

8. The dilator of claim 1, wherein the dilator further comprises a tip portion located on the distal end portion of the shaft, and the tip portion includes a first exit port and a second exit port in fluid communication with the flushing lumen.

9. The dilator of claim 1, wherein the flushing lumen is a first flushing lumen and the dilator further comprises a second flushing lumen,
wherein the entrance port is a first entrance port in fluid communication with the first flushing lumen, and
the dilator further comprises a second entrance port in the proximal end portion of the shaft, the second entrance port configured to be in fluid communication with the introducer sheath and in fluid communication with the second flushing lumen.

10. The dilator of claim 9, wherein the exit port is a first exit port in fluid communication with the first flushing lumen, and
the dilator further comprises a second exit port located in the distal end portion of the shaft and in fluid communication with the second flushing lumen.

11. The dilator of claim 9, wherein the dilator further comprises a tip portion located on the distal end portion of the shaft,
the tip portion includes a first exit port in fluid communication with the first flushing lumen, and
the tip portion includes a second exit port in fluid communication with the second flushing lumen.

12. The dilator of claim 9, wherein the dilator has a central axis, and
the first flushing lumen is located opposite the second flushing lumen along the central axis.

13. The dilator of claim 1, further comprising a central lumen extending longitudinally through the shaft from the proximal end portion to the distal end portion.

14. The dilator of claim 13, wherein the central lumen is configured to receive a guidewire therethrough.

15. A dilator configured for use with an introducer sheath, the dilator comprising:
a shaft including a proximal end portion and a distal end portion;
a flushing lumen extending longitudinally through the shaft from the proximal end portion to the distal end portion, and configured for injecting a liquid through the dilator;
an entrance port in the proximal end portion of the shaft, the entrance port configured to be in fluid communication with the introducer sheath and in fluid communication with the flushing lumen; and
a central lumen extending longitudinally through the shaft from the proximal end portion to the distal end portion.

16. The dilator of claim 15 wherein the central lumen is configured to receive a guidewire therethrough.

17. The dilator of claim 15, wherein the entrance port is configured to be in fluid communication with a flushing port of the introducer sheath.

18. The dilator of claim 15, further comprising a lock located on the proximal end portion of the shaft and configured to engage the introducer sheath and couple the dilator to the introducer sheath.

19. The dilator of claim 15, further comprising a hub located on the proximal end portion of the shaft and including a lock configured to engage the introducer sheath and couple the dilator to the introducer sheath.

20. The dilator of claim 15, further comprising an exit port located in the distal end portion of the shaft and in fluid communication with the flushing lumen.

21. The dilator of claim 15, further comprising a first exit port and a second exit port located in the distal end portion of the shaft and in fluid communication with the flushing lumen.

22. The dilator of claim 15, wherein the dilator further comprises a tip portion located on the distal end portion of the shaft, and the tip portion includes an exit port in fluid communication with the flushing lumen.

23. The dilator of claim 15, wherein the dilator further comprises a tip portion located on the distal end portion of the shaft, and the tip portion includes first and second exit ports in fluid communication with the flushing lumen.

24. The dilator of claim 15, wherein the flushing lumen is a first flushing lumen and the dilator further comprises a second flushing lumen,
wherein the entrance port is a first entrance port in fluid communication with the first flushing lumen, and
the dilator further comprises a second entrance port in the proximal end portion of the shaft, the second entrance port configured to be in fluid communication with the introducer sheath and in fluid communication with the second flushing lumen.

25. The dilator of claim 24, wherein the exit port is a first exit port in fluid communication with the first flushing lumen, and
the dilator further comprises a second exit port located in the distal end portion of the shaft and in fluid communication with the second flushing lumen.

26. The dilator of claim 24, wherein the dilator further comprises a tip portion located on the distal end portion of the shaft,
the tip portion includes a first exit port in fluid communication with the first flushing lumen, and
the tip portion includes a second exit port in fluid communication with the second flushing lumen.

27. The dilator of claim 24, wherein the dilator has a central axis, and
the first flushing lumen is located opposite the second flushing lumen along the central axis.

28. A dilator configured for use with an introducer sheath, the dilator comprising:
a shaft including a proximal end portion and a distal end portion;
first and second flushing lumens extending longitudinally through the shaft from the proximal end portion to the distal end portion, and configured for injecting a liquid through the dilator;
first and second entrance ports in the proximal end portion of the shaft, the first and second entrance ports configured to be in fluid communication with the introducer sheath and in fluid communication with the first and second flushing lumens respectively; and
a central lumen extending longitudinally through the shaft from the proximal end portion to the distal end portion.

29. The dilator of claim 28, wherein the central lumen is configured to receive a guidewire therethrough.

30. The dilator of claim 28, wherein the first and second entrance ports are configured to be in fluid communication with a flushing port of the introducer sheath.

31. The dilator of claim 28, further comprising a lock located on the proximal end portion of the shaft and configured to engage the introducer sheath and couple the dilator to the introducer sheath.

32. The dilator of claim 28, further comprising a hub located on the proximal end portion of the shaft and including a lock configured to engage the introducer sheath and couple the dilator to the introducer sheath.

33. The dilator of claim 28, further comprising first and second exit ports located in the distal end portion of the shaft and in fluid communication with the first and second flushing lumens respectively.

34. The dilator of claim 28, wherein the dilator further comprises a tip portion located on the distal end portion of the shaft, and the tip portion includes first and second exit ports in fluid communication with the first and second flushing lumens respectively.

35. The dilator of claim 28, wherein the dilator has a central axis, and
the first flushing lumen is located opposite the second flushing lumen along the central axis.

36. A method of using a dilator comprising:
introducing a dilator into an introducer sheath; and
injecting a liquid into an injection port of the introducer sheath and through a lumen of the dilator.

37. The method of claim 36, further comprising coupling the dilator to the introducer sheath.

38. The method of claim 36, wherein the liquid is a contrast media.

39. The method of claim 36, further comprising introducing a guidewire into a lumen of the dilator.

40. A method of manufacturing a dilator configured for use with an introducer sheath, the method comprising:
forming a shaft including a proximal end portion and a distal end portion;
forming a flushing lumen into the shaft extending longitudinally through the shaft from the proximal end portion of the shaft to the distal end portion of the shaft; and
forming an entrance port in the proximal end portion of the shaft and in fluid communication with the flushing lumen, the entrance port configured to be in fluid communication with an injection port of the introducer sheath.

41. The method of claim 40, further comprising sealing a proximal end of the flushing lumen.

42. The method of claim 41, wherein sealing the proximal end of the flushing lumen includes sealing the proximal end of the flushing lumen with epoxy.

43. The method of claim 40, further comprising forming an exit port located in the distal end portion of the shaft and in fluid communication with the flushing lumen.

44. The method of claim 40, further comprising forming a tip portion on the distal end portion of the shaft; and
forming an exit port in the tip portion of the shaft and in fluid communication with the flushing lumen.

45. The method of claim 40, wherein forming a flushing lumen into the shaft includes forming first and second flushing lumens.

46. The method of claim 45, further comprising sealing a proximal end of the first and second flushing lumens.

47. The method of claim 46, wherein sealing the proximal ends of the first and second flushing lumens includes sealing the proximal ends of the flushing lumens with epoxy.

48. The method of claim 45, wherein forming an entrance port includes forming first and second entrance ports in fluid communication with the first and second flushing lumens respectively, the first and second entrance ports configured to be in fluid communication with an injection port of the introducer sheath.

49. The method of claim 45, further comprising forming first and second exit ports located in the distal end portion of the shaft and in fluid communication with the first and second flushing lumens respectively.

50. The method of claim 45, further comprising forming a tip portion on the distal end portion of the shaft; and
forming first and second exit ports in the tip portion of the shaft and in fluid communication with the first and second flushing lumens respectively.

51. The method of claim 40, further comprising forming a central lumen into the shaft.

52. The method of claim 51, wherein the central lumen is configured to receive a guidewire therethrough.

53. The method of claim 40, further comprising coupling a hub to the proximal end portion of the shaft.

54. The method of claim 40, further comprising coupling a lock to the proximal end portion of the shaft,
wherein the lock is configured to engage the introducer sheath and couple the dilator to the introducer sheath.
